(19) United States
(12) Patent Application Publication
(10) Pub. No.: US 2026/0047877 A1
(43) Pub. Date: Feb. 19, 2026

(54) ELECTROSURGICAL SYSTEMS AND METHODS FACILITATING MONOPOLAR AND/OR BIPOLAR ELECTROSURGICAL TISSUE TREATMENT

(71) Applicant: Covidien LP, Mansfield, MA (US)

(72) Inventors: James E. DUNNING, Boulder, CO (US); Kelly N. Kneale, Boulder, CO (US)

(73) Assignee: Covidien LP, Mansfield, MA (US)

(21) Appl. No.: 19/102,160

(22) PCT Filed: Aug. 1, 2023
(86) PCT No.: PCT/IB2023/057787

§ 371 Date: Feb. 7, 2025

Related U.S. Application Data
(60) Provisional application No. 63/395,912, filed on Aug. 8, 2022.

(51) Int. Cl. A61B 18/12; A61B 18/00

(52) U.S. Cl. CPC A61B 18/1206; A61B 2018/00642; A61B 2018/00702; A61B 2018/00755; A61B 2018/00767; A61B 2018/00892; A61B 2018/1253; A61B 2018/126

(57) ABSTRACT

An electrosurgical system includes an electrosurgical generator configured to output electrosurgical energy to an electrosurgical device for application to tissue. The electrosurgical generator is configured to determine a condition of contact between at least one electrode of the electrosurgical device and tissue or a surgical technique of using the electrosurgical device and to control the electrosurgical energy output by the electrosurgical generator based on the determined condition or surgical technique.

### FIELD

The present disclosure relates to electrosurgery and, more particularly, to electrosurgical systems and methods facilitating monopolar and/or bipolar electrosurgical tissue treatment.

### BACKGROUND

A monopolar electrosurgical device typically includes an active electrode configured to deliver energy from an electrosurgical generator to tissue. The monopolar electrosurgical device is utilized in conjunction with a return electrode device, e.g., a remote return pad or localized return electrode, that completes the electrical circuit between the electrosurgical generator and the patient. Such monopolar electrosurgical devices include probes, hooks, loops, etc.

A bipolar electrosurgical device typically includes an active electrode and a return electrode that are electrically isolated from one another and charged to different potentials to enable the flow of current between the active and return electrodes and through intervening tissue to complete the electrical circuit between the electrosurgical generator and the patient. Such bipolar electrosurgical devices include probes, forceps, scissors, etc.

In both monopolar and bipolar electrosurgery, electrical current is conducted through tissue to heat tissue to achieve a desired tissue treatment, e.g., to coagulate and/or cut tissue.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

Provided in accordance with the present disclosure is an electrosurgical system including an electrosurgical generator configured to output electrosurgical energy to at least one electrode of an electrosurgical device for application to tissue. The electrosurgical generator includes sensor circuitry configured to sense electrical data associated with the application of energy to tissue and a controller having a processor and non-transitory computer readable storage medium storing instructions that, when executed by the processor, cause the controller to obtain the sensed electrical data, determine a condition of contact between the at least one electrode of the electrosurgical device and tissue based on the sensed electrical data, and control the electrosurgical energy output by the electrosurgical generator based on the determined condition.

In an aspect of the present disclosure, the condition of contact is an open circuit condition. In such aspects, controlling the electrosurgical energy output may include decreasing a voltage of the electrosurgical energy output.

In another aspect of the present disclosure, the condition of contact is entering or exiting an open circuit condition. In such aspects, controlling the electrosurgical energy output may include decreasing a voltage of the electrosurgical energy output.

In still another aspect of the present disclosure, the condition of contact is a single electrode contact condition. In such aspects, controlling the electrosurgical energy output may include stopping or pausing the electrosurgical energy output.

Another electrosurgical system provided in accordance with aspects of the present disclosure includes an electrosurgical generator configured to output electrosurgical energy to at least one electrode of an electrosurgical device for application to tissue. The electrosurgical generator includes sensor circuitry configured to sense electrical data associated with the application of energy to tissue or activation data associated with activation of the application of energy to tissue, and a controller. The controller has a processor and non-transitory computer readable storage medium storing instructions that, when executed by the processor, cause the controller to obtain the electrical data or the activation data, determine a surgical technique performed using the electrosurgical device based on the electrical data or the activation data, and control the electrosurgical energy output by the electrosurgical generator based on the determined surgical technique.

In an aspect of the present disclosure, the electrosurgical generator is configured to output monopolar electrosurgical energy to a monopolar electrosurgical device.

In another aspect of the present disclosure, the surgical technique is a buzzing the hemostat technique. In such aspects, controlling the electrosurgical energy output may include increasing power and/or minimizing impedance.

In yet another aspect of the present disclosure, the electrosurgical generator configured to output bipolar electrosurgical energy to a bipolar electrosurgical device.

In still another aspect of the present disclosure, determining the surgical technique includes performing pattern recognition on the activation data associated with the activation of the application of energy to tissue.

In still yet another aspect of the present disclosure, the surgical technique is a spot coagulation technique. In such aspects, controlling the electrosurgical energy output may include increasing voltage and/or increasing power.

Yet another electrosurgical system provided in accordance with aspects of the present disclosure includes an electrosurgical generator configured to output electrosurgical energy to an electrosurgical device for application to tissue. The electrosurgical generator includes sensor circuitry configured to sense electrical data associated with the application of energy to tissue and a controller having a processor and non-transitory computer readable storage medium storing instructions that, when executed by the processor, cause the controller to obtain an activation command to initiate the output of the electrosurgical energy, initially control the output of the electrosurgical energy to achieve rapid desiccation of tissue, and subsequently control the output of the electrosurgical energy based on the sensed electrical data to increase power and decrease tissue impedance.

In an aspect of the present disclosure, the controller is further caused, during the subsequent controlling, to monitor for a spike in the sensed electrical data. Monitoring for a spike in the sensed electrical data may include monitoring for a spike in at least one of voltage or crest factor.

In another aspect of the present disclosure, the controller is further caused to decrease a power of the electrosurgical energy in response to detecting a spike in the sensed electrical data while monitoring for the spike in the sensed electrical data.

In another aspect of the present disclosure, the electrosurgical generator is configured to output bipolar electrosurgical energy to a bipolar electrosurgical device including first and second electrodes configured to conduct electrosurgical energy therebetween.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a schematic illustration of a monopolar electrosurgical system provided in accordance with aspects of the present disclosure shown in use treating tissue of a patient;

FIG. 1B is a schematic illustration of a bipolar electrosurgical system provided in accordance with aspects of the present disclosure shown in use treating tissue of a patient;

FIG. 2 is a front view of the electrosurgical generator of the systems of FIGS. 1A and 1B;

FIG. 3 is a block diagram of the electrosurgical generator of the systems of FIGS. 1A and 1B;

FIGS. 4-6 are block diagrams of methods facilitating electrosurgical tissue treatment in accordance with aspects of the present disclosure; and

FIG. 7 is a flow diagram of another method facilitating electrosurgical tissue treatment in accordance with aspects of the present disclosure.

### DETAILED DESCRIPTION

The present disclosure provides electrosurgical systems and methods facilitating monopolar and/or bipolar electrosurgical tissue treatment such as, for example, by adjusting energy delivery based on feedback to achieve a desired tissue effect while minimizing adverse effects such as sparking and charring of tissue. Exemplary monopolar and bipolar electrosurgical systems are detailed hereinbelow; however, it is understood that the aspects and features of the present disclosure are equally applicable for use with any suitable monopolar and/or bipolar electrosurgical systems, including multi-modality systems, robotic systems, etc.

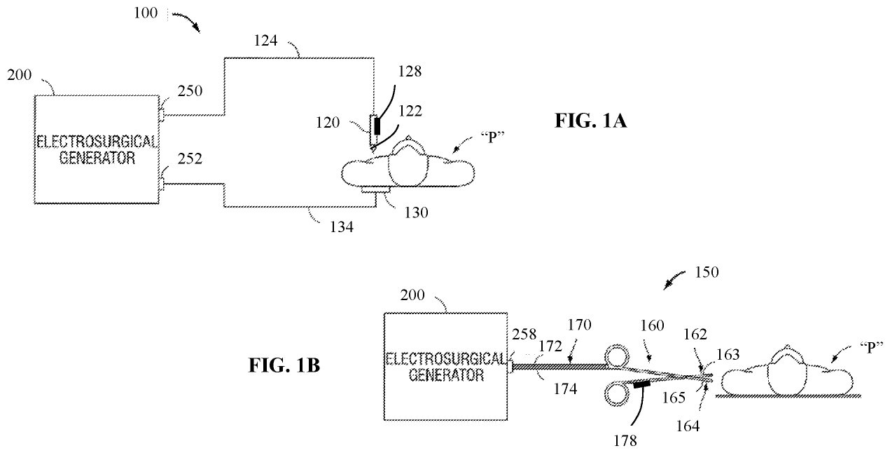

Referring to FIG. 1A, a monopolar electrosurgical system provided in accordance with aspects of the present disclosure is shown generally identified by reference numeral 100. Monopolar electrosurgical system 100 is configured to selectively apply monopolar electrosurgical energy, e.g., monopolar radio frequency (RF) energy, to target tissue of a patient “P.” System 100, more specifically, includes a monopolar electrosurgical device 120, a return electrode device 130, and an electrosurgical generator 200. Monopolar electrosurgical device 120 includes at least one active electrode 122 configured to supply energy to tissue of a patient “P.” Monopolar electrosurgical device 120 may be, for example, a monopolar electrosurgical pencil, a monopolar electrosurgical knife, a monopolar electrode loop, a monopolar electrode of a multi-function device, a monopolar ablation device, a surgical robotic monopolar end effector, or any other suitable device including an active electrode 122 configured to supply energy to tissue of a patient “P.” Monopolar electrosurgical device 120 further includes an electrosurgical cable 124 having a plug (not explicitly shown) configured to connect active electrode 122 to an active monopolar port 250 of electrosurgical generator 200. Return electrode device 130 likewise includes an electrosurgical cable 134 having a plug (not explicitly shown) configured to connect to a return monopolar port 252 of electrosurgical generator 200. In this manner, monopolar electrosurgical energy can be supplied from electrosurgical generator 200 to active electrode 122 for application to tissue and return to electrosurgical generator 200 via return device 130.

Monopolar electrosurgical device 120 also includes one or more activation controls 128 disposed thereon. Alternatively or additionally, one or more activation controls may be disposed on a remote activation device, e.g., a footswitch, on electrosurgical generator 200, or otherwise provided to facilitate activation and/or control of monopolar electrosurgical device 120. Each of the one or more activation controls 128 may be configured as a push button, slider, toggle switch, dial, trigger, virtual button, touch-screen interface, etc. Each of the one or more activation controls 128 is configured to connect to electrosurgical generator 200, e.g., via one or more wires extending through cable 124 to enable a surgeon to selectively activate and/or control monopolar electrosurgical device 120. Further, each of the one or more activation controls 128 may be configured to enable at least one of: activation of the supply of energy to active electrode 122, deactivation of the supply of energy to active electrode 122, selection of a mode of operation, selection of a power level, etc.

System 100 may be utilized to supply monopolar electrosurgical energy to tissue, for example, to coagulate or cut tissue, although other tissue treatments are also contemplated. Further, monopolar electrosurgical device 120 may be configured to operate in a plurality of different modes corresponding to different power levels, different tissue treatments, different types of tissue to be treated, different surgical techniques utilized, different surgical conditions, etc.

Turning to FIG. 1B, a bipolar electrosurgical system provided in accordance with aspects of the present disclosure is shown generally identified by reference numeral 150. Bipolar electrosurgical system 150 is configured to selectively apply bipolar electrosurgical energy to tissue of a patient “P.” System 150 includes a bipolar electrosurgical device 160 and electrosurgical generator 200. Bipolar electrosurgical device 160 may be, for example, a bipolar electrosurgical forceps (as detailed herein), although other suitable bipolar electrosurgical devices are contemplated such as, for example, bipolar electrosurgical forceps, bipolar electrosurgical probes, bipolar electrosurgical resection loops, bipolar electrosurgical scissors, surgical robotic bipolar end effectors, etc. Bipolar electrosurgical device 160 includes opposing first and second jaw members 162, 164 including first and second electrodes 163, 165, respectively. First and second electrodes 163, 165 are configured to connect to bipolar port 258 of electrosurgical generator 200 by way of a cable 170 having a plug (not explicitly shown) and including first and second electrode leads 172, 174 that connect to respective first and second electrodes 163, 165. First and second electrode leads 172, 174 are electrically isolated from one another and connect to different pins of the plug of cable 170 to thereby connect to the active and return terminals of bipolar port 258 of electrosurgical generator 200. In this manner, bipolar electrosurgical energy is supplied from electrosurgical generator 200 to bipolar electrosurgical device 160 via cable 170, conducted between first and second electrodes 163, 165 and through tissue, e.g., tissue grasped between first and second jaw members 162, 164, and is returned to electrosurgical generator 200 via cable 170.

Bipolar electrosurgical device 160 also includes one or more activation controls 178 disposed thereon. Alternatively or additionally, one or more activation controls may be disposed on a remote activation device, e.g., a footswitch, on electrosurgical generator 200, or otherwise provided to facilitate activation and/or control of bipolar electrosurgical device 160. Each of the one or more activation controls 178 may be configured as a push button, slider, toggle switch, dial, trigger, virtual button, touch-screen interface, etc. Each of the one or more activation controls 178 is configured to connect to electrosurgical generator 200, e.g., via one or more wires extending through cable 170 to enable a surgeon to selectively activate and/or control bipolar electrosurgical device 160. Further, each of the one or more activation controls 178 may be configured to enable at least one of: activation of the supply of energy to bipolar electrosurgical device 160, deactivation of the supply of energy to bipolar electrosurgical device 160, selection of a mode of operation, selection of a power level, etc.

System 150 may be utilized to supply bipolar electrosurgical energy to tissue, for example, to coagulate or cut tissue, although other tissue treatments are also contemplated. Further, bipolar electrosurgical device 160 may be configured to operate in a plurality of different modes corresponding to different power levels, different tissue treatments, different types of tissue to be treated, different surgical techniques utilized, different surgical conditions, etc.

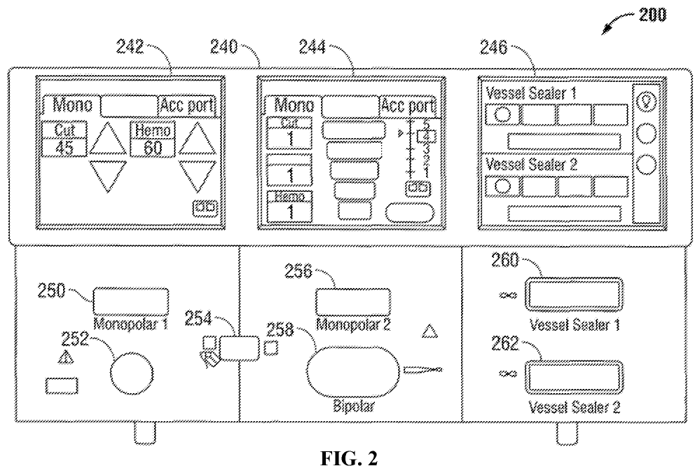

Turning to FIG. 2, a front face 240 of electrosurgical generator 200 is shown. Electrosurgical generator 200 may include a plurality of ports 250-262 to accommodate various types of electrosurgical devices (e.g., monopolar electrosurgical device 120, bipolar electrosurgical device 160, etc.) and/or accessories (e.g., foot switches, remote User Interfaces (UI's), communication hubs, robotic systems, etc.). One or more of ports 250-262 may include a detection device configured to read information stored or otherwise encoded on the plugs of devices connected thereto (e.g., the plugs of cables 130, 170 of devices 120, 150 (FIGS. 1A and 1B, respectively)) to obtain identifying information, authentication information, use information, and/or operating parameters of the devices connected to electrosurgical generator 200, thus enabling electrosurgical generator 200 to selectively enable and configure energy delivery and energy delivery settings based on the information pertaining to the connected device(s). Such detection devices may be configured to read information from bar codes, electrical components (e.g., resistors, capacitors, etc.), RFID chips, magnets, non-transitory storage (e.g., non-volatile memory, EEPROM, etc.), etc., which may be coupled to or integrated into the plugs of the devices. Suitable detection devices include, for example, bar code readers, electrical sensors, RFID readers, Hall Effect sensors, memory readers, and/or any other suitable decoders configured to read information.

Continuing with reference to FIG. 2, electrosurgical generator 200 includes one or more display screens 242, 244, 246 for providing the user with a variety of output information (e.g., activation settings, mode settings, intensity settings, treatment complete indicators, feedback information (tissue impedance, for example), etc.). Each of screens 242, 244, 246 may be associated with a corresponding port 250-262 or ports 250-262. Electrosurgical generator 200 further includes suitable activation controls (e.g., buttons, activators, switches, touch screen, etc.) for controlling electrosurgical generator 200 and/or the devices connected thereto. The display screens 242, 244, 246 may further be configured as touch screens that display a corresponding menu for the electrosurgical device(s) connected to electrosurgical generator, thereby facilitating selection of settings and/or modes of operation for those particular device(s).

Ports 250-262 may include, for example: one or more active monopolar device ports 250, 256 each configured to receive a plug of a monopolar electrosurgical device, e.g., monopolar electrosurgical device 120 (FIG. 1A); a return monopolar device port 252 configured to receive a plug of a monopolar electrosurgical return device, e.g., return device 130 (FIG. 1A); an input device port 254, e.g., such as for a footswitch; a bipolar device port 258 configured to receive a plug of a bipolar electrosurgical device, e.g., bipolar electrosurgical device 160 (FIG. 1B); first and second vessel sealing ports 260, 262 each configured to receive a plug of a vessel sealing device; and/or any other suitable ports.

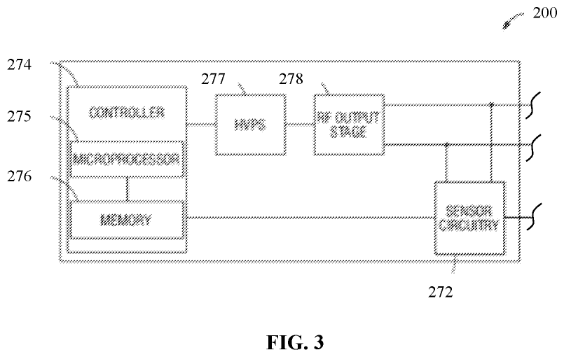

Turning to FIG. 3, electrosurgical generator 200 includes sensor circuitry 272, a controller 274, a high voltage DC power supply (“HVPS”) 277, and an RF output stage 278. HVPS 277 provides high voltage DC power to RF output stage 278 which converts the high voltage DC power into RF energy for delivery to the connected device(s), e.g., monopolar electrosurgical device 120 (FIG. 1A) and/or bipolar electrosurgical device 160 (FIG. 1B). In particular, RF output stage 278 generates waveforms of high frequency RF energy. RF output stage 278 is configured to generate a plurality of waveforms having various duty cycles, peak voltages, crest factors, and other parameters, depending on particular settings and/or modes of operation. With respect to outputting monopolar energy, e.g., to active electrode 122 of monopolar electrosurgical device 120 (FIG. 1A), RF output stage 278 is configured to output the high frequency RF energy to cable 124 for delivery to active electrode 122 (see FIG. 1A) while energy is returned via cable 134 of return device 130 (FIG. 1A). With respect to outputting bipolar energy, e.g., to electrodes 163, 165 of bipolar electrosurgical device 160 (FIG. 1B), RF output stage 278 is configured to output the high frequency RF energy to leads 172, 174 of cable 170 of bipolar electrosurgical device 160 (FIG. 1B).

Controller 274 includes a microprocessor 275 (or other suitable processor such as, for example, a digital signal processor, an ASIC, a graphics processing unit (GPU), a field-programmable gate array (FPGA), or a central processing unit (CPU)) operably connected to a memory 276 which may be volatile type memory (e.g., RAM) and/or non-volatile type memory (e.g., flash media, disk media, etc.). Microprocessor 275 is operably connected to HVPS 277 and/or RF output stage 278 allowing microprocessor 275 to control the output of electrosurgical generator 200, e.g., in accordance with feedback received from sensor circuitry 272. Sensor circuitry 272 is operably coupled to the energy supply leads that supply energy to/from tissue, e.g., to/from monopolar electrosurgical device 120 and return device 130 in monopolar configurations (FIG. 1A) and to/from electrodes 163, 165 of bipolar electrosurgical device 160 (FIG. 1B) in bipolar configurations. From these leads and, more specifically, the signals transmitted therealong, sensor circuitry 272 may determine one or more parameters, e.g., tissue impedance, current, voltage (rms voltage and/or peak voltage), power, crest factor, and/or phase. Sensor circuitry 272 provides feedback, e.g., based on the sensed parameter(s), to controller 274 which, in turn, selects an energy-delivery algorithm, modifies an energy-delivery algorithm, and/or adjusts energy-delivery parameters based thereon. More specifically, controller 274 may be configured to control power, voltage (rms voltage and/or peak voltage), current, tissue impedance, crest factor, and/or phase of the output electrosurgical energy based on the feedback received from sensor circuitry 272 and according to an implemented energy-delivery algorithm or algorithms. As noted above, electrosurgical generator 200 may also read information from the connected device(s) to enable control based on the type, configuration, and/or settings of the connected device(s).

Sensor circuitry 272 and/or controller 274 may also monitor the wires connecting activation controls, e.g., activation controls 128 (FIG. 1A) and/or activation controls 178 (FIG. 1B), and/or other inputs to electrosurgical generator 200 to determine activation, deactivation, settings, modes, and/or other operational inputs and to control electrosurgical generator 200 based thereon. Alternatively or additionally, sensor circuitry 272 and/or controller 274 may be configured to monitor other sensors associated with monopolar electrosurgical device 120, bipolar electrosurgical device 160, electrosurgical generator 200, and/or other devices. For example, an accelerometer associated with one or more of the devices may provide feedback regarding motion of the device from which usage, technique, and/or intent may be determined.

In aspects, memory 276 can be separate from controller 274 and communicate with microprocessor 275 through communication buses of a circuit board, through communication cables such as serial ATA cables or other types of cables, and/or via suitable wireless communication protocols. Regardless of the location(s) of memory 276 and/or microprocessor 275, memory 276 includes computer-readable instructions that are executable by microprocessor 275 to operate controller 274, e.g., for executing various algorithms such as, for example, fixed algorithms, machine learning algorithms, etc. Controller 274 may further include a network interface (not shown) to communicate with other computers or a server. In aspects, a storage device (not shown) of controller 274 or separate therefrom may be used for storing data.

Although illustrated as part of electrosurgical generator 200, it is also contemplated that controller 274 be remote from electrosurgical generator 200, e.g., on a remote server, and accessible by electrosurgical generator 200 via a wired or wireless connection. In configurations where controller 274 is remote, it is contemplated that controller 274 may be accessible by and connected to multiple electrosurgical generators 200.

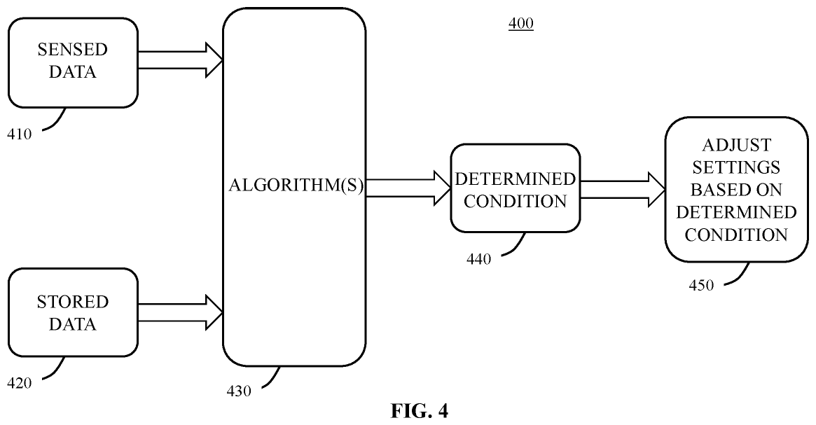

Turning to FIGS. 4-7, electrosurgical methods facilitating monopolar and/or bipolar electrosurgical tissue treatment are described. To the extent consistent, any of the methods detailed herein with respect to monopolar electrosurgery may also be utilized with respect to bipolar electrosurgery and vice versa. Further, the electrosurgical methods detailed herein are not mutually exclusive; rather, to the extent consistent, any or all of the electrosurgical methods detailed herein may be utilized in any suitable combination implemented simultaneously, serially, alternatingly, in overlapping temporal relation, or in any other suitable manner. The electrosurgical methods detailed herein may be implemented by monopolar electrosurgical system 100 (FIG. 1A), bipolar electrosurgical system 150 (FIG. 1B), and/or any other suitable electrosurgical system. In particular, in aspects, the processing and control detailed below may be performed by controller 274 of electrosurgical generator 200 (FIG. 3).

Referring to FIG. 4, method 400 is configured for use during application of energy, e.g., monopolar or bipolar electrosurgical energy, to tissue. Method 400 includes obtaining sensed data 410 and, in aspects, stored data 420, and providing the data 410, 420 to one or more algorithms 430 to determine a condition associated with the application of energy 440. Energy delivery settings may then be adjusted based on the determined condition 450 as necessary.

In aspects, the sensed data 410 includes electrical feedback data, e.g., from sensor circuitry 272 (FIG. 3), such as, for example, power, voltage (rms voltage and/or peak voltage), current, tissue impedance, crest factor, and/or phase of the output electrosurgical energy. Other suitable sensed data 410 is also contemplated.

The stored data 420 may include information relating to the device(s) in use (e.g., obtained from one of the detection devices of electrosurgical generator 200 (FIG. 2)), prior electrical feedback data, and/or prior activation data. The stored data 420 may additionally or alternatively include information to be utilized by the one or more algorithms such as, for example: operating thresholds, operating ranges, coefficients, formulas, tables, etc.

The one or more algorithms 430 may include: algorithms comparing the sensed data 410 and/or the stored data 420 against set points or ranges; algorithms retrieving information from one or more look-up tables based on the sensed data 410 and/or the stored data 420; algorithms configured as one or more functions (e.g., linear, polynomial, piece-wise, or other suitable functions) that receive, as input variables, the sensed data 410 and/or the stored data 420; machine learning algorithm, etc.

With respect to machine learning algorithms, the algorithms may be trained using training data that is obtained experimentally, via mathematical simulation, utilizing machine learning, using theoretical formulae, combinations thereof, etc. The machine learning algorithm(s) may be trained and then deployed for use or may be continually trained and updated based on local and/or remote (from other systems) data. In aspects, training the machine learning algorithm(s) may be performed by the electrosurgical generator or may be performed by a computing device or devices separate from the electrosurgical generator and then the resulting algorithm may be communicated to the electrosurgical generator.

In aspects, the one or more machine learning algorithms may include classification machine learning and/or one or more neural networks such as, for example, a long-short term memory network, a convolutional neural network (CNN), a recurrent adversarial network (RAN), a generative adversarial network (GAN) and/or other suitable neural network(s). As an alternative or in addition to a neural network, other suitable machine learning systems may be utilized such as, for example a support vector machine (SVM), and/or may implement: Bayesian Regression, Naive Bayes, nearest neighbors, least squares, means, and support vector regression, among other data science and artificial science techniques. Other suitable machine learning algorithms are also contemplated.

Continuing with reference to FIG. 4, the determined condition 440 may be, for example, an open circuit condition. For example, in monopolar surgery, an open circuit condition is present where the device is activated without (e.g., prior to) contact between the active electrode and tissue. An alternative or additional determined condition 440 may be entering or exiting an open circuit condition. For example, in monopolar surgery, entering or exiting an open circuit condition occurs where the device is activated with minimal contact or reducing contact (e.g., beginning or removing contact) between the active electrode and tissue. With respect to these open circuit or entry/exit open circuit conditions 440, adjusting the settings based on the determined condition 450 may include, for example, decreasing (e.g., ramping down) the voltage of the supplied energy so that sparking into tissue upon entry/exit (e.g., upon initiating contact with tissue and/or removal from contact with tissue) is eliminated or minimized.

Where the determined condition 440 is a normal operating condition (e.g., not an open circuit condition or an entry/exit open circuit condition), adjusting the settings based on the determined condition 450 may include maintaining power to facilitate tissue treatment, e.g., tissue dissection. So long as the normal operating condition persists, power can be safely maintained, e.g., via increasing voltage as necessary, even in high-impedance tissue, to achieve the desired tissue treatment. For example, such a configuration enables efficient and effective tissue cutting even through high-impedance tissue.

Continuing with reference to FIG. 4, in aspects, the determined condition 440 is a single electrode contact condition. For example, in bipolar surgery, a single electrode contact condition is present where the device is activated with only one of the electrodes maintaining sufficient contact with tissue. It has been found that a single electrode contact condition results in higher and more erratic impedances during the application of energy to tissue whereas, with respect to normal operating conditions (where both electrodes are in sufficient contact with tissue prior to activation), relatively smooth and consistent voltage and current plots and lower impedance are realized. The sensed data 410 for detecting a single electrode contact condition may include impedance data sampled for a pre-determined period of time, e.g., 0.02 s, once a pre-determined power threshold has been reached, e.g., 1 W. The one or more algorithms 430 may be configured to perform time integration on the impedance data and, based on the results, determine whether a single electrode contact condition exists by comparing the result to a threshold value (which may be stored data 420). Other suitable algorithms 430 are also contemplated.

Where a single electrode contact condition is determined to exist, adjusting the settings based on the determined condition 450 may include momentarily pausing the supply of energy, stopping the supply of energy (thus requiring re-activation), and/or outputting a warning to the user, e.g., from electrosurgical generator 200.

Referring still to FIG. 4, in aspects, the determined condition 440 may be an impedance condition of tissue. For example, in monopolar surgery, tissue in contact with the active electrode may be classified as low impedance tissue or not low impedance tissue, although additional or alternative impedance classifications are also contemplated. Where it is determined that the active electrode is in contact with low impedance tissue, adjusting the settings based on the determined condition 450 may include decreasing the voltage to eliminate or minimize sparking and/or charring of tissue. Such a configuration enables precise tissue treatment, e.g., dissection, even in low impedance tissue.

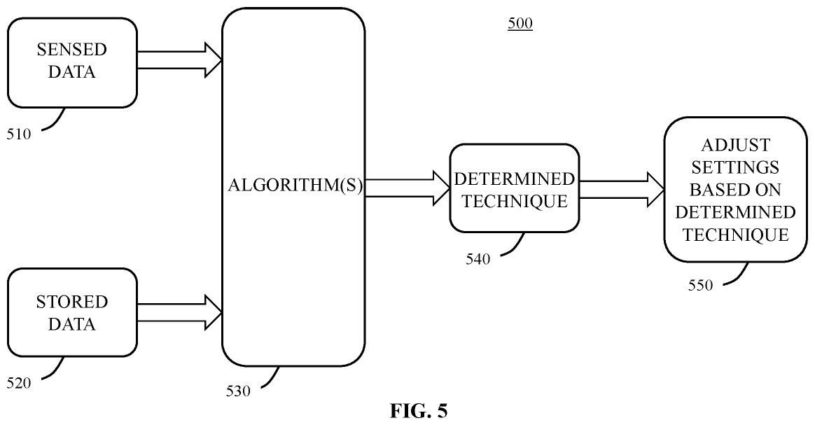

Turning to FIG. 5, method 500 is configured for use during application of energy, e.g., monopolar or bipolar electrosurgical energy, to tissue. Method 500 includes obtaining sensed data 510 and, in aspects, stored data 520, and providing the data 510, 520 to one or more algorithms 530 to determine a surgical technique being performed 540. Energy delivery settings may then be adjusted based on the determined technique 550 as necessary.

In aspects, the sensed data 510 includes electrical feedback data, e.g., from sensor circuitry 272 (FIG. 3), such as, for example, power, voltage (rms voltage and/or peak voltage), current, tissue impedance, crest factor, and/or phase of the output electrosurgical energy. Other suitable sensed data 510 is also contemplated such as, for example, sensed data from other sensors and/or devices. More specifically, in aspects, accelerometer or other motion data from one or more devices may be utilized. In aspects, video and/or audio data from a respective camera and/or microphone incorporated into one or more of the devices or separate therefrom may be utilized. This additional sensed data may be utilized to facilitate determination of usage, technique, intent, tissue type, tissue condition, environmental condition(s), etc.

The stored data 520 may include information relating to the device(s) in use (e.g., obtained from one of the detection devices of electrosurgical generator 200 (FIG. 2)), prior electrical feedback data, and/or prior activation data. The stored data 520 may additionally or alternatively include information to be utilized by the one or more algorithms such as, for example: operating thresholds, operating ranges, coefficients, formulas, tables, etc.

The one or more algorithms 530 may include: algorithms comparing the sensed data 510 with the stored data 520; algorithms retrieving information from one or more look-up tables based on the sensed data 510; algorithms configured as one or more functions (e.g., linear, polynomial, piece-wise, or other suitable functions) that receive, as input variables, the sensed data 510 and/or the stored data 520; pattern recognition algorithms, machine learning algorithms (such as detailed above or other suitable machine learning algorithms), etc.

The determined technique 540 may be, for example, buzzing the hemostat. Buzzing the hemostat is a surgical technique whereby tissue is grasped with a grasping hemostat or forceps and the active electrode of a monopolar electrosurgical device is moved into contact with the bare metal of the hemostat or forceps to energize the hemostat or forceps and enable coagulation of the grasped tissue. However, the settings associated with the monopolar electrosurgical device may impact the effectiveness of buzzing the hemostat. By employing the one or more algorithms 530, method 500 enables the determination of whether the surgeon is using the buzz the hemostat technique and, based thereon, adjustment of settings to facilitate treatment using the buzz the hemostat technique.

Relatively low impedance and a relatively clean current waveform may indicate that a surgeon is utilizing the buzz the hemostat technique, whereas relatively high impedance and a relatively noisy current waveform may indicate the surgeon is directly treating tissue with the monopolar electrosurgical device. These realizations may be utilized, for example, in establishing the one or more algorithms 530 configured to detect whether the buzz the hemostat technique is being used.

Where a buzz the hemostat technique is determined, adjusting the settings based on the determined technique 550 may include implementing and/or tightening safety thresholds (of power, impedance, voltage, and/or current, for example). Alternatively or additionally, adjusting the settings based on the determined technique 550 may include monitoring the electrical data and adjusting power to maximize power and minimize impedance. Such may also include monitoring for spikes in voltage or crest factor that would indicate sparking and/or charring of tissue. Where a buzz the hemostat technique is not (or is no longer) determined, the energy delivery settings may revert to default settings.

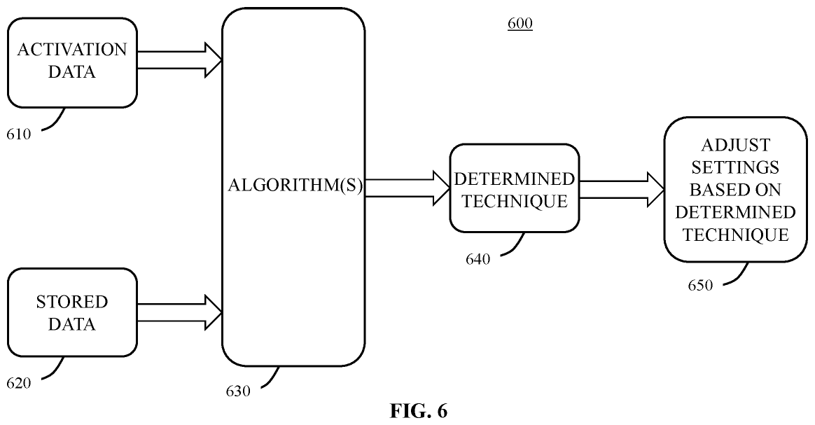

With reference to FIG. 6, method 600 is configured for use during application of energy, e.g., monopolar or bipolar electrosurgical energy, to tissue. Method 600 includes obtaining activation data 610 and, in aspects, stored data 620, and providing the data 610, 620 to one or more algorithms 630 to determine a surgical technique being performed 640. Energy delivery settings may then be adjusted based on the determined technique 650 as necessary.

In aspects, the activation data 610 includes a number, sequence, and/or pattern of activations of one or more activation controls associated with the device in use, e.g., activation controls 128 (FIG. 1A) and/or activation controls 178 (FIG. 1B).

The stored data 620 may include information relating to the device(s) in use (e.g., obtained from one of the detection devices of electrosurgical generator 200 (FIG. 2)), prior electrical feedback data, and/or prior activation data. The stored data 620 may additionally or alternatively include information to be utilized by the one or more algorithms such as, for example: operating thresholds, operating ranges, coefficients, formulas, tables, etc.

The one or more algorithms 630 may include: algorithms comparing the sensed data 610 with the stored data 620; algorithms retrieving information from one or more look-up tables based on the sensed data 610; algorithms configured as one or more functions (e.g., linear, polynomial, piece-wise, or other suitable functions) that receive, as input variables, the sensed data 610 and/or the stored data 620; pattern recognition algorithms, machine learning algorithms (such as detailed above or other suitable machine learning algorithms), etc.

Continuing with reference to FIG. 6, in aspects, the determined technique 640 is spot coagulation. The spot coagulation technique may be determined based on pattern recognition (machine learning-based or based on traditional algorithm(s)) of a repetitive pattern of short bursts of energy (as reflected in the activation data 610 via repeated activation and deactivation). Upon detecting the spot coagulation technique, adjusting the settings based on the determined technique 650 may include increasing voltage and power output to facilitate spot coagulation. Where the spot coagulation technique is not detected or is no longer detected, the settings may remain unchanged or may revert to default settings. This configuration enables effective and efficient spot coagulation.

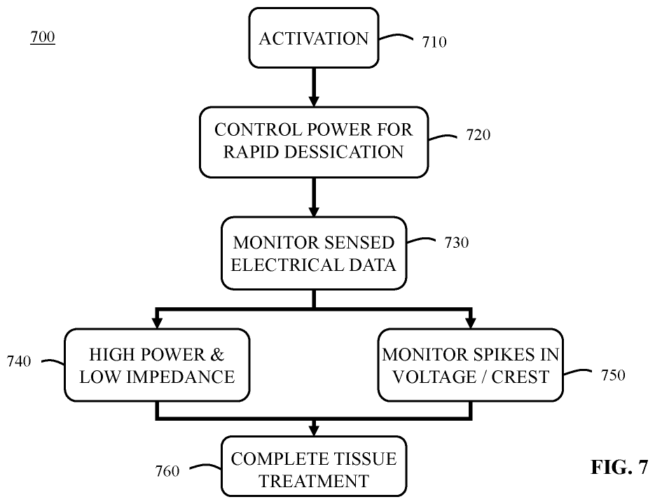

Turning to FIG. 7, another method provided in accordance with the present disclosure is shown generally identified as method 700. Method 700 may be configured for use during application of energy to tissue. For example, method 700 may be implemented with respect to bipolar electrosurgical energy delivery in a coagulation mode, although other implementations of method 700 are also contemplated. With respect to bipolar coagulation, performance is enhanced at lower impedance, e.g., approximately 50 to 125 ohms. However, the optimal power setting is difficult to pre-set because the tissue effect is slow in the beginning of activation and, after tissue has been desiccated to a certain degree, the tissue effect can be undesirable, e.g., causing over-desiccation, charring, and/or sparking. Thus, instead of a pre-defined power setting, method 700 controls power to efficiently and effectively achieve the desired tissue effect, e.g., coagulation.

Initially, at 710, an activation input, e.g., from an activation control (such as activation control 178 (FIG. 1B)), is received to initiate the supply of energy to tissue. In response, energy is applied to tissue whereby power is controlled to facilitate rapid desiccation of tissue, as indicated at 720. This may be accomplished, for example, by monitoring sensed data as indicated at 730, e.g., monitoring current, rms voltage, peak voltage, crest factor, impedance, phase, and/or time components thereof (such as rate of change of voltage or current), to achieve rapid desiccation of tissue, thereby speeding up the initial tissue effect. Once desiccation is achieved, the sensed data is continually monitored and the output adjusted to achieve and maintain high power and low impedance, as indicated at 740. At the same time, the sensed data is monitored for spikes in voltage or crest factor, as indicated at 750. Spikes in voltage or crest factor may indicate sparking and, thus, in response to detecting such spikes, power may be reduced and/or other energy settings adjusted to inhibit or minimize sparking and charring of tissue. The monitoring and control as indicated at 730-750 continued until tissue treatment is complete, as indicated at 760, e.g., until energy application is deactivated.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. An electrosurgical system, comprising:
an electrosurgical generator configured to output electrosurgical energy to at least one electrode of an electrosurgical device for application to tissue, the electrosurgical generator including:
sensor circuitry configured to sense electrical data associated with the application of energy to tissue; and
a controller having a processor and non-transitory computer readable storage medium storing instructions that, when executed by the processor, cause the controller to:
obtain the sensed electrical data;
determine a condition of contact between the at least one electrode of the electrosurgical device and tissue based on the sensed electrical data; and
control the electrosurgical energy output by the electrosurgical generator based on the determined condition.

2. The electrosurgical system according to claim 1, wherein the condition of contact is an open circuit condition.

3. The electrosurgical system according to claim 2, wherein controlling the electrosurgical energy output includes decreasing a voltage of the electrosurgical energy output.

4. The electrosurgical system according to claim 1, wherein the condition of contact is entering or exiting an open circuit condition.

5. The electrosurgical system according to claim 5, wherein controlling the electrosurgical energy output includes decreasing a voltage of the electrosurgical energy output.

6. The electrosurgical system according to claim 1, wherein the condition of contact is a single electrode contact condition.

7. The electrosurgical system according to claim 6, wherein controlling the electrosurgical energy output includes stopping or pausing the electrosurgical energy output.

8. An electrosurgical system, comprising:
an electrosurgical generator configured to output electrosurgical energy to at least one electrode of an electrosurgical device for application to tissue, the electrosurgical generator including:
sensor circuitry configured to sense electrical data associated with the application of energy to tissue or activation data associated with activation of the application of energy to tissue; and
a controller having a processor and non-transitory computer readable storage medium storing instructions that, when executed by the processor, cause the controller to:
obtain the electrical data or the activation data;
determine a surgical technique performed using the electrosurgical device based on the electrical data or the activation data; and
control the electrosurgical energy output by the electrosurgical generator based on the determined surgical technique.

9. The electrosurgical system according to claim 8, wherein the electrosurgical generator is configured to output monopolar electrosurgical energy to a monopolar electrosurgical device and wherein the sensor circuitry is configured to sense the electrical data associated with the application of energy to tissue.

10. The electrosurgical system according to claim 9, wherein the surgical technique is a buzzing the hemostat technique.

11. The electrosurgical system according to claim 10, wherein controlling the electrosurgical energy output includes at least one of increasing power or minimizing impedance.

12. The electrosurgical system according to claim 8, wherein the electrosurgical generator configured to output bipolar electrosurgical energy to a bipolar electrosurgical device and wherein the sensor circuitry is configured to sense the activation data associated with the activation of the application of energy to tissue.

13. The electrosurgical system according to claim 12, wherein determining the surgical technique includes performing pattern recognition on the activation data associated with the activation of the application of energy to tissue.

14. The electrosurgical system according to claim 12, wherein the surgical technique is a spot coagulation technique.

15. The electrosurgical system according to claim 14, wherein controlling the electrosurgical energy output includes at least one of increasing voltage or increasing power.

16. An electrosurgical system, comprising:
an electrosurgical generator configured to output electrosurgical energy to an electrosurgical device for application to tissue, the electrosurgical generator including:
sensor circuitry configured to sense electrical data associated with the application of energy to tissue; and
a controller having a processor and non-transitory computer readable storage medium storing instructions that, when executed by the processor, cause the controller to:
obtain an activation command to initiate the output of the electrosurgical energy;
initially control the output of the electrosurgical energy to achieve rapid desiccation of tissue; and
subsequently control the output of the electrosurgical energy based on the sensed electrical data to increase power and decrease tissue impedance.

17. The electrosurgical system according to claim 16, wherein the controller is further caused, during the subsequent controlling, to monitor for a spike in the sensed electrical data.

18. The electrosurgical system according to claim 17, wherein monitoring for a spike in the sensed electrical data includes monitoring for a spike in at least one of voltage or crest factor.

19. The electrosurgical system according to claim 17, wherein the controller is further caused to decrease a power of the electrosurgical energy in response to detecting a spike in the sensed electrical data while monitoring for the spike in the sensed electrical data.

20. The electrosurgical system according to claim 16, wherein the electrosurgical generator is configured to output bipolar electrosurgical energy to a bipolar electrosurgical device including first and second electrodes configured to conduct electrosurgical energy therebetween.
